(19) United States
(12) Patent Application Publication
(10) Pub. No.: US 2012/0301413 A1
(43) Pub. Date: Nov. 29, 2012

(54) EXTERNAL COMPOSITION FOR SKIN CONTAINING MANASSANTIN B AS ACTIVE INGREDIENT AND THE USE THEREOF FOR SKIN WHITENING

(75) Inventors: Hui Kyoung CHANG, Yongin-si (KR); DaeGun KIM, Seoul (KR); Myeong Hoon YEOM, Yongin-si (KR); Hyun Jung CHOI, Suwon-si (KR); Kyung Mi JOO, Suwon-si (KR); Jae Sung HWANG, Seoul (KR); Soo Mi AHN, Suwon-si (KR)

(73) Assignee: AMOREPACIFIC CORPORATION, Seoul (KR)

(21) Appl. No.: 13/568,975

(22) Filed: Aug. 7, 2012

Related U.S. Application Data
(63) Continuation of application No. 12/515,151, filed on Jun. 22, 2010.

(30) Foreign Application Priority Data
Nov. 17, 2006 (KR) 10-2006-0113662

(51) Int. Cl. A61K 8/49; A61Q 19/02

(52) U.S. Cl. 424/62

(57) ABSTRACT

Disclosed herein are an external composition for skin, which contains, as an active ingredient, manassantin B extracted from Saururus chinensis Baill, and the use thereof for skin whitening. More particularly, disclosed are an external composition for skin, which contains, as an active ingredient, manassantin B, which inhibits the transfer of melanosomes, thus providing a skin-whitening effect without influencing the melanin synthesis function of the melanocytes, as well as the use thereof for skin whitening.

### TECHNICAL FIELD

The present invention relates to an external composition for skin, which contains, as an active ingredient, manassantin B extracted from Saururus chinensis Baill, and to the use thereof for skin whitening. More particularly, the present invention relates to an external composition for skin, which contains, as an active ingredient, manassantin B which inhibits the binding between two melanosome transporter proteins, melanophilin and myosin 5a, in melanocytes, to inhibit the migration of melanin, thus exhibiting an excellent skin-whitening effect, as well as the use thereof for skin whitening.

### BACKGROUND ART

The understanding of the chemical and enzymatic of melanogenesis as a mechanism, which determines the color of the skin, is well documented. Melanocytes synthesize melanin in the organelle melanosomes, which are then transferred to keratinocyte dendrites through keratinocytes. Melanin transferred to keratinocytes in this manner is an important factor which actually determines the skin color. In transferring melanosomes to melanocyte dendrites, major constituents are involved. They are called “MTC” (melanosome transport complex)”, which consists of three proteins, melanophilin, Rab27a and myosin 5a. Rab27a serves as a transporter, which binds to the surface of melanin so as to recognize and transport melanosome. In order for Rab27a to migrate to the periphery, it must meet the myosin 5a protein as the intracellular cytoskeleton, and a protein serving as an adapter to recognize and connect the two proteins to each other is the melanophilin protein. When the three constituents meet each other to form a complex, it is possible to normally transfer melanosomes to the periphery, and ultimately to keratinocytes. If any one of the three constituents is abnormal, it is observed that a problem arises in the transport of melanosomes without causing any problem in the production of melanin, so that the color of the skin becomes white. Therefore, a skin whitening effect can be obtained by inhibiting the transfer of melanosomes through the regulation of these constituents.

Saururus chinensis Baill is a perennial plant like Houttuynia cordata and has various pharmacological effects. It is known to have a significant effect on the prevention and treatment of various adult diseases, including constipation, diabetes, liver diseases, cancers, hypertension, heart diseases, women's diseases and renal diseases. It is known that flavonoid substances, including quercetin, quercitrin, isoquercitrin and rutin, which are active ingredients found in Saururus chinensis Baill, all function to clean blood more potently than the flavonoid substances of ginkgo leaves and protect and strengthen peripheral arteries which supply clean blood evenly to each tissue of the body, and water-soluble tannin has the effect of preventing DNA mutation, and thus shows a great ability to inhibit the development of cancer.

In pharmacological studies on extracts isolated from Saururus chinensis Baill, Korean Patent Laid-Open publication No. 96-21001 discloses that the extract is used in a cosmetic composition for the prevention of aging (wrinkles) in order to employ the antioxidant effect (radical elimination effect of the above-described flavonoid substances (Korean Patent Laid-Open publication No. 96-21001). In addition, Korean Patent Laid-Open publication No. 2002-0035656 discloses the Saururus chinensis Baill extract is used in a cosmetic composition for skin whitening in order to employ the effect of inhibiting the activity of tyrosinase, a melanin-producing enzyme, even though the active ingredients of the extract, found through precise isolation and purification, are not described.

Manassantin B was reported to inhibit the activity of acyl CoA cholesterol acyltransferase (Korean Patent Application No. 10-2003-0080397), but there is no report on the skin-whitening effect of this substance itself.

### DISCLOSURE

### Technical Problem

The present inventors have found for the first time that, when melan-a cells as melanocytes are treated with a Saururus chinensis Baill extract, the transfer of melanosomes is inhibited. Also, through the isolation and purification of the Saururus chinensis Baill extract, the present inventors have identified for the first time that an active ingredient performing this function is manassantin B. Furthermore, the present inventors have found that the active ingredient manassantin B has the effect of inhibiting the transfer of melanosomes by inhibiting the binding between transporter proteins performing the transfer of melanosomes in melanocytes, and thus it can have a skin whitening effect, even if melanin synthesis is normal.

Accordingly, it is an object of the present invention to provide a skin external composition for skin whitening, which contains manassantin B, a Saururus chinensis Baill extract, and thus has the effect of inhibiting the transfer of melanosomes in melanocytes.

### Technical Solution

To achieve the above object, in one aspect, the present invention provides an external composition for skin, which comprises manassantin B as an active ingredient.

Said manassantin B is characterized in that it is isolated and purified from Saururus chinensis Baill.

In another aspect, the present invention provides the use of composition for skin, which comprises manassantin B as an active ingredient, for skin whitening.

Hereinafter, the present invention will be described in further detail.

Melanosome of melanocytes is an intracellular organelle producing melanin having a function of blocking cell damage caused by UV rays and is a very important factor, because this organelle must be transferred to keratinocytes such that cell damage caused by UV rays can be minimized.

Manassantin B inhibits the transfer of melanosomes by inhibiting the binding between melanophilin and myosin 5a, which are transporter proteins performing the transfer of melanosomes in melanocytes, and thus it provides a skin-whitening effect, even if melanin synthesis is normal.

The skin external composition according to the present invention contains, as an active ingredient, manassantin B in an amount of 0.0001-10 wt %, and preferably 0.001-10 wt %, based on the total weight of the composition, in view of the effect, safety and formulation stability of the composition. This is because, if the content of manassantin B is less than 0.001 wt %, the effect thereof cannot be expected, and if the content of manassantin B exceeds 10 wt %, it will be difficult to ensure the safety and formulation stability of the composition.

The composition of the present invention preferably has a formulation selected from among solution, suspension, emulsion, paste, gel, cream, lotion, powder, soap, surfactant-containing cleansing, oil, powder foundation, emulsion foundation, wax foundation and spray formulations.

The skin external composition containing manassantin B can be used for skin whitening.

### Advantageous Effects

As described above, when cells are treated with manassantin B isolated from Saururus chinensis Baill, melanosome aggregation, which appears when the transfer of melanosomes in melan-a cells as melanocytes is inhibited, can be observed, and this results from the inhibition of binding between melanophilin and myosin 5a proteins as melanosome transporter proteins. Accordingly, manassantin B interferes with the binding between melanophilin and myosin 5a proteins as melanosome transporter proteins, and thus it can provide a skin whitening effect by inhibiting the transfer of melanosomes without influencing the melanin synthesis function of the melanosomes.

### DESCRIPTION OF DRAWINGS

FIG. 1 illustrates photographs showing the results of optical microscope observation for the configuration of melanocyte (melan-a) cells after treatment with manassantin B.

FIG. 2 is a graphic diagram showing the degree of inhibition of binding between Mlph (melanophilin) and myosin 5a.

FIG. 3 is a graphic diagram showing that manassantin B of the present invention does not inhibit the activity of tyrosinase at the cell level.

FIG. 4 is a graphic diagram showing that manassantin B of the present invention inhibits the synthesis of melanin in a concentration-dependent manner in the co-culture of melanocytes with keratinocytes.

FIG. 5 is a graph obtained by making an artificial pigmented macule on the back of guinea pigs, applying a sample to the macule for one week, visually observing whether the color of the artificial pigmented macule applied with the sample became lighter than that of an artificial pigmented macule not treated with anything (untreated group), and expressing the observation results as numerical values.

FIG. 6 shows L values obtained by measuring the color of the artificial pigmented macule with a colorimeter after one week of treatment with the sample.

### BEST MODE

Hereinafter, the present invention will be described in further detail with reference to the following examples and test examples, but the scope of the present invention is not limited only to these examples.

### Example 1

### Preparation of Saururus chinensis Baill Extract

1 kg of Saururus chinensis Baill (whole plant) was added to 5 l of 70% ethanol (water, a water-containing organic solvent (ethanol, methanol, butanol, ether, ethyl acetate, chloroform, methylene chloride or the like) or an organic solvent) and extracted three times under reflux. Then, the solution was left to stand for 15° C. for 1 day. Then, the solution was filtered through filer cloth and centrifuged into the filtrate and the residue, and the separated filtrate was concentrated under reduced pressure. The resulting extract was suspended in 1 l of water, and then extracted five times with 1 l of methylene chloride. The total methylene chloride layer thus obtained was concentrated under reduced pressure, thus obtaining 50 g of Saururus chinensis Baill extract.

### Example 2

### Analysis of Structure of Manassantin B

Step 1: Purification of Manassantin B

10 g of the Saururus chinensis Baill extract obtained in Example 1 was purified through silica gel column chromatography (packed with 300 g of silica gel). As developing solvents, hexane and ethyl acetate were used, the concentration gradient of hexane to ethyl acetate was increased from 5:1 to 1:5 to obtain fractions. From these fractions, 0.62 g of manassantin B was obtained.

Step 2: Analysis of Manassantin B Structure by NMR

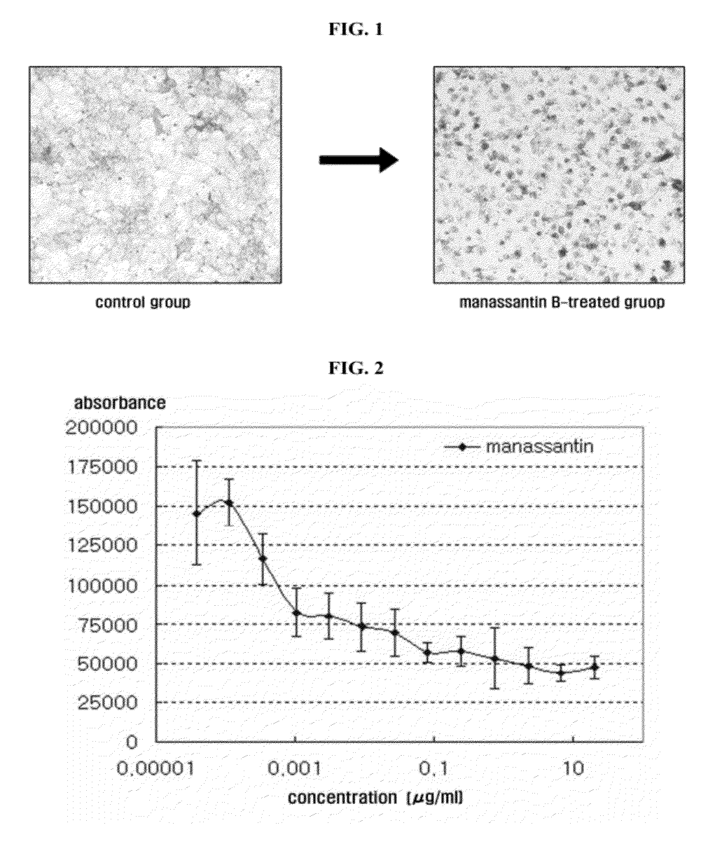

The product obtained in the step 1 was analyzed with an NMR spectrometer. As a result, data shown in Table 1 below were obtained, and the products showed the following properties. Thus, the product was identified to be manassantin B and had a structure represented by the following chemical FIG. 1:

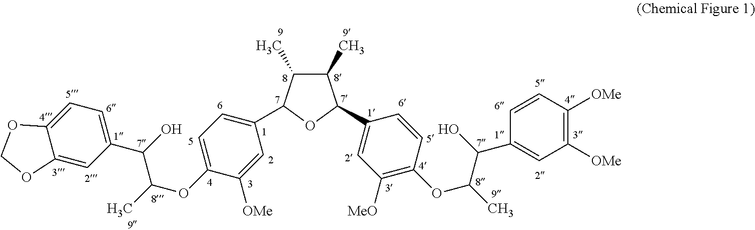

### <Physical and Chemical Properties of Manassantin B>

Properties: light cream-colored fine crystals

Positive FAB-MS: 734.5 (M+H₂O)

->LC-MS (APCI, positive): 734.5 (M+H₂O)

TABLE 1
¹H and ¹³C-NMR data for manassantin B

| Carbon | δ_{H} | δ_{C} | dept |
|---|---|---|---|
| 1 | — | 136.8 | C |
| 2 | 6.94 | 112.3 | CH |
| 3 | — | 151.6 | C |
| 4 | — | 147.9 | C |
| 5 | 6.98(dd, 8.49) | 118.0 | CH |
| 6 | 6.83(dd, 8.05) | 120.2 | CH |
| 7 | 5.45(d, 6.32) | 85.5 | CH |
| 8 | 2.31(ddq) | 44.9 | CH |
| 9 | 0.68(d, 5.0) | 14.9 | CH₃ |
| 1′ | — | 136.9 | C |
| 2′ | 6.94 | 112.3 | CH |
| 3′ | — | 151.7 | C |
| 4′ | — | 147.9 | C |
| 5′ | 7.00(dd, 8.49) | 118.5 | CH |
| 6′ | 6.83(dd, 8.05) | 120.2 | CH |
| 7′ | 5.45(d, 6.32) | 85.5 | CH |
| 8′ | 2.31(ddq) | 44.9 | CH |
| 9′ | 0.67(d, 5.0) | 14.9 | CH₃ |
| 1″ | — | 135.3 | C |
| 2″ | 7.05(s) | 112.3 | CH |
| 3″ | — | 150.4 | C |
| 4″ | — | 150.2 | C |
| 5″ | 6.92 | 112.7 | CH |
| 6″ | 6.98 | 121.1 | CH |
| 7″ | 4.68(d, 6.29) | 78.1 | CH |
| 8″ | 4.41(dq) | 81.7 | CH |
| 9″ | 1.08(d, 5.0) | 16.6 | CH₃ |
| 1′″ | — | 136.4 | C |
| 2′″ | 6.89 | 108.7 | CH |
| 3′″ | — | 148.7 | C |
| 4′″ | — | 149.1 | C |
| 5′″ | 6.83 | 108.9 | CH |
| 6′″ | 6.91 | 121.9 | CH |
| 7′″ | 4.66(d, 6.43) | 78.2 | CH |
| 8′″ | 4.41(dq) | 81.9 | CH |
| 9′″ | 1.07(d, 5.0) | 16.6 | CH₃ |
| 3′″-OCH2O-4′″ | 5.92(s) | 102.4 | CH₂ |
| 3-OCH3 | 3.87(s) | 56.7 | CH₃ |
| 3′-OCH3 | 3.87(s) | 56.7 | CH₃ |
| 3″-OCH3 | 3.81(s) | 56.5 | CH₃ |
| 4″-OCH3 | 3.82(s) | 56.6 | CH₃ |

### Test Example 1

### Examination of Effect of Manassantin B on Inhibition of Melanosome Transfer at Cell Level

Step 1: Culture of Melanocytes

Melan-a cells, immortalized mouse melanocytes, were cultured to a confluency of 90% in 10% fetal bovine serum-containing RPMI 1640 medium in a 10% CO₂ incubator, while the medium was replaced with fresh medium at 3-day intervals.

Step 2: Examination of Inhibition of Melanin Transfer in Melanocytes by Manassantin B

The melan-a cells cultured in the step 1 were separated using 0.25% trypsin-EDTA and spread on a 6-well plate at a density of 3×10⁵ cells/well. Then, the cells were left to stand for 24 hours. The cells were treated with manassantin B at various concentrations, and after 4 days, the configuration of the cells was observed with an optical microscope. The observation results are shown in FIG. 1.

As can be seen from parts looking black in FIG. 1, in the cells treated with manassantin B, melanosomes were gathered together around the nuclei without being evenly distributed throughout the cells, unlike a control group not treated with manassantin B.

### Test Example 2

### Examination of Inhibition of Binding Between Melanophilin and Myosin 5a by Manassantin B

Step 1: Preparation of Recombinant Protein

Proteins required in the test were produced in large amounts using a suitable system (E. coli) or baculovirus. Herein, the designed proteins essentially included domains known to be required for protein interaction and contained GST, His tag or flag tag such that the produced proteins could be easily purified and isolated. Each of a histidine tagged-GFP-melanophilin recombinant protein and a Flag-MyosinVa recombinant protein, which were to be used as bait proteins, were expressed in a baculovirus system, cells containing the proteins were disrupted, and then extracts were prepared. Flag-MyosinVa GT was prepared into an extract with PBS TG buffer solution, and then purified by flag-affinity chromatography.

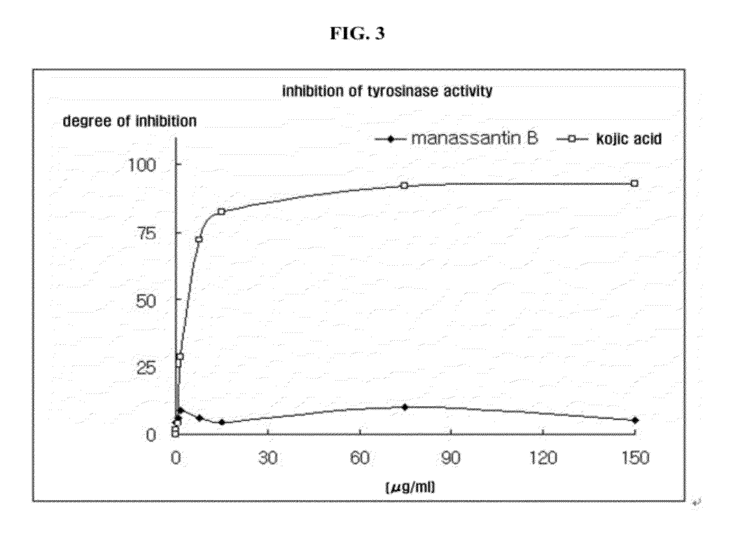

Step 2: Protein Binding Between Melanophilin and Myosin 5a The his-GFP-mlphCT protein prepared in the step 1 was bound to a nickel-coated plate through reaction. To the plate, binding partner Flag-MyosinVa GT was bound, and then an M2 antibody recognizing the flag was bound. The plate was subjected to color development reaction by chemiluminescence to measure the degree of binding between the proteins. The measurement results are shown in FIG. 2.

As can be seen in FIG. 2, manassantin B inhibited the binding between melanophilin and myosin 5a.

### Test Example 3

### Observation of Tyrosinase Activity Inhibitory Effect of Manassantin B

Step 1: Culture of Melanocytes

Melanocytes were cultured in the same manner as in the step 1 of Test Example 1.

Step 2: Measurement of Inhibition of Tyrosinase Activity The cells cultured in the step 1 were washed once with cold PBS, and then disrupted with 0.1M phosphate buffer (containing 1% triton x-100). Then, the cells were centrifuged at 1400 rpm for 20 minutes, and the cell supernatant was isolated. 40 μg of the protein thus prepared was mixed with various concentrations of manassantin B and allowed to react for 1 hour. The reaction material was treated with 2 mg/ml of L-DOPA solution and left to stand 37° C. After 15 minutes, the absorbance at 490 nm was measured, and the measurement results are shown in FIG. 3.

As can be seen in FIG. 3, manassantin B did not directly inhibit the activity of tyrosinase.

### Test Example 4

### Examination of Inhibition of Melanin Synthesis by Manassantin B

Step 1: Coculture of Melanocytes and Keratinocyts

Melanocytes were cultured in the same manner as in the step 1 of Test Example 1. Mouse keratinocytes SP-1 cells were cultured to a confluency of 90% in 8% fetal bovine serum-containing s-MEM medium in a 10% CO₂ incubator, while the medium was replaced with fresh medium at 3-days intervals.

Step 2: Measurement of Inhibition of Melanin Synthesis

The melan-a cells and SP-1 cells cultured in the step 1 were separated with 0.25% Trypsin-EDTA and spread together on a 12-well plate at a ratio of 1:10 at cell densities of 1×10⁴ cells/well for melan-a and 1×10⁵ cells/well for SP-1. Then, the cells were left to stand for 24 hours. The cells were treated with manassantin B at various concentrations, and after 5 days, the cells were collected with 1N NaOH. The collected cells were measured for absorbance at 405 nm and measured for protein concentration using the Lowry method. The measurement results are shown in FIG. 4.

As can be seen in FIG. 4, manassantin B inhibited melanin synthesis in a concentration-dependent manner in the coculture of melanocytes and keratinocytes, and the synthesis of melanin was inhibited by about 50% at a manassantin B concentration of 0.02 ppm.

### Test Example 5

### Examination of Whitening Effect of Manassantin B Through Animal Experiment

Step 1: Preparation of Samples to be Used in Animal Experiment

The extract prepared in Example 2 was dissolved in vehicle (ethylene: propylene glycol: water=5:3:2) to a concentration of 0.1%, and kojic acid was dissolved in vehicle at a concentration of 5%.

Step 2: Animal Experiment Using Brown Guinea Pigs for Whitening Effect of Manassantin B

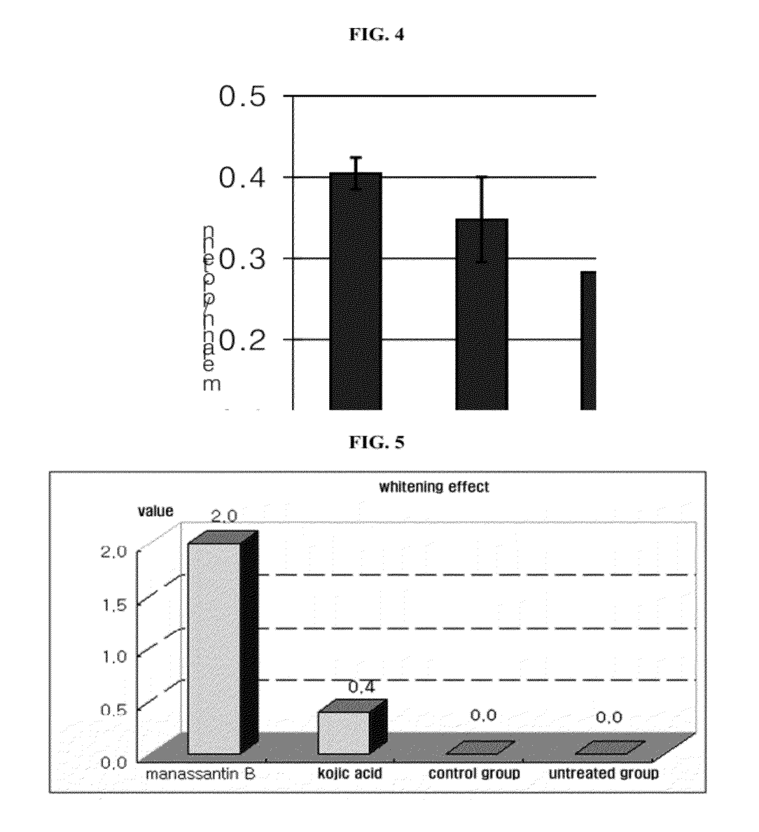

The hair of the back of guinea pigs weighing about 500 g was shaved, and the samples prepared in the step 1 were applied to the shaved skin in a circle shape having a diameter of about 1 cm twice a day in the morning and evening for two days. At day 3, the guinea pigs were anesthetized with pentobarbital (30 mg/kg), and then the circle portions applied with the samples were irradiated with 250 mJ of UV light. From the next day of UV irradiation, the same substances as applied at the first day were applied twice in the morning and evening for 1 week. Then, the difference in the color of an artificial pigmented macule, treated only with vehicle, and an artificial pigmented macule, treated with the samples, from an artificial pigmented macule not applied with anything (untreated group), was visually observed. In the visual observation, 0.5 indicates a whitening effect which can be recognized only by specialists; 1, a whitening effect which can be recognized by everyone; 3, a whitening effect showing a lightness corresponding to the original skin color; 2, a whitening effect between 1 and 3; and 4, a whitening effect showing a lightness higher than the original skin color. Four animals for each group were used in the experiment, and the obtained numerical values were averaged and graphed. A higher numerical value shows that the artificial pigmented macule became white by application with the samples, and the observation results are shown in FIG. 5.

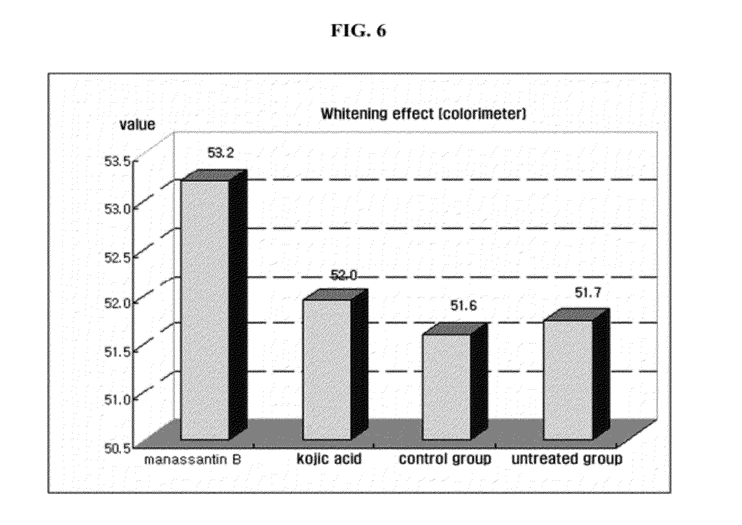

In addition to the above visual observation, the lightness of the artificial pigmented macula was evaluated using a colorimeter, and the results were expressed as numerical values (L values) and averaged. The averaged numerical values are graphically shown in FIG. 6, in which a higher numerical value indicates a lighter color.

As can be seen in FIG. 6, the manassantin B-containing extract showed an excellent whitening effect compared to the control group.

### Formulation Example 1

### Lotion Containing Manassantin B

Components 1-6 in Table 2 below were mixed with each other and dissolved at 70 r to obtain an aqueous phase.

Then, components 8-14 were dissolved at 70 t to obtain an oil phase. Then, the aqueous phase together with component 7 was added to the oil phase, and the mixture was stirred in a homo-mixer (Tokushu Kika, Japan) to obtain an emulsion. Then, the emulsion was thickened by the addition of component 15. After removing bubbles, the resulting material was cooled to room temperature, thus preparing lotion.

TABLE 2

| Components | Content (wt %) |
|---|---|
| 1. Purified water | To 100 |
| 2. Glycerin | 8.0 |
| 3. Butylene glycol | 4.0 |
| 4. Hyaluronic acid extract | 5.0 |
| 5. Beta-glycan | 7.0 |
| 6. Carbomer | 0.1 |
| 7. Manassantin B | 0.1 |
| 8. Caprylic/capric triglyceride | 8.0 |
| 9. Squalane | 5.0 |
| 10. Cetearyl glucoside | 1.5 |
| 11. Sorbitan stearate | 0.4 |
| 12. Cetearyl alcohol | 1.0 |
| 13. Preservative | q.s. |
| 14. Fragrance | q.s. |
| 15. Triethanolamine | 0.1 |

### Formulation Example 2

### Nourishing Cream Containing Manassantin B

Nourishing cream having the components and contents shown in Table 3 below was prepared.

TABLE 3

| Components | Content (wt %) |
|---|---|
| Purified water | To 100 |
| Glycerin | 3.0 |
| Butylene glycol | 3.0 |
| Liquid paraffin | 7.0 |
| Beta-glucan | 7.0 |
| Carbomer | 0.1 |
| Manassantin B | 1.0 |
| Caprylic/capric triglyceride | 3.0 |
| Squalane | 5.0 |
| Cetearyl glucoside | 1.5 |
| Sorbitan stearate | 0.4 |
| Polysorbate 60 | 1.2 |
| Preservative | q.s. |
| Fragrance | q.s. |
| Pigment | q.s. |
| Triethanolamine | 0.1 |

### Formulation Example 3

### Pack Containing Containing Manassantin B

A pack having the components and contents shown in Table 4 below was prepared.

TABLE 4

| Components | Contents (wt %) |
|---|---|
| Purified water | To 100 |
| Glycerin | 4.0 |
| Polyvinyl alcohol | 15.0 |
| Hyaluronic acid extract | 5.0 |
| Beta-glucan | 7.0 |
| Allantoin | 0.1 |
| Manassantin B | 0.2 |
| Nonyl phenylether | 0.4 |
| Polysorbate 60 | 1.2 |
| Preservative | q.s. |
| Fragrance | q.s. |
| Pigment | q.s. |
| Ethanol | 6.0 |

### Test Example 6

### Examination of Whitening Effect of Manassantin B Through Human Body Experiment

12 healthy men were used as test subjects. An opaque tape through which 6 holes having a diameter of 1.5 cm were formed was attached to the upper arm of each subject, and then each subject was exposed to UV light (UVB) at about 1.5-2 times the minimal erythema dose to induce the darkening of the skin and applied with the test substances. After 2 months, the lightness of the skin was measured with a chromameter. As the test substances, the compositions obtained in Example 1, Formulation Example 1 and Comparative Example were applied to each subject twice a day in the morning and evening. Herein, Comparative Example is the same composition as that of Formulation Example 1, except that it contained no manassantin B.

The effects of the test substances were determined by measuring “L value” indicating the lightness of the skin (the color of the non-tanned skin of Koreans generally shows an L value of 50-70). A gradual increase in L value indicates that the test substance is effective, and the comparison between the test substances was expressed as ΔL value. Herein, ΔL=final L value—value before application of test substance. A higher ΔL value indicates a greater whitening effect. The test results are shown in Table 5 below.

TABLE 5

| | Formulation Example 1 | Example 1 | Comparative Example |
|---|---|---|---|
| ΔL | 2.25 | 2.89 | 1.04 |

As shown in Table 5, the compositions containing manassantin B showed ΔL values of 2.25 and 2.89, respectively, which were higher than 1.04 for Comparative Example. This suggests that the compositions containing manassantin B had an excellent whitening effect.

### INDUSTRIAL APPLICABILITY

As described above, the inventive skin external composition containing manassantin B extracted from Saururus chinensis Baill can provide a skin whitening effect by inhibiting the transfer of malanosomes.

1-6. (canceled)

7. A method for whitening skin of a mammal comprising applying manassantin B to the skin of the mammal.

8. The method of claim 7, wherein said manassantin B is isolated from Saururus chinensis Baill extract.

9. The method of claim 7, wherein said manassantin B is contained in a composition in an amount of 0.001-10 wt % based on the total weight of the composition.

10. The method of claim 9, wherein the composition further contains kojic acid and a vehicle.

11. A method for whitening skin of a mammal comprising applying a composition consisting essentially of manassantin B to the skin of the mammal.

12. The method of claim 11, wherein the composition further contains kojic acid and a vehicle.

13. The method of claim 11, wherein said manassantin B is isolated from Saururus chinensis Baill extract.

14. The method of claim 11, wherein said manassantin B is contained in a composition in an amount of 0.001-10 wt % based on the total weight of the composition.
